(19) United States
(12) Patent Application Publication
(10) Pub. No.: US 2011/0144722 A1
(43) Pub. Date: Jun. 16, 2011

(54) MRI-COMPATIBLE IMPLANTABLE LEAD WITH IMPROVED LC RESONANT COMPONENT

(75) Inventors: Xiaoyi Min, Thousand Oaks, CA (US); Ingmar Viohl, Canyon Country, CA (US)

(73) Assignee: PACESETTER, INC., Sylmar, CA (US)

(21) Appl. No.: 12/635,340

(22) Filed: Dec. 10, 2009

(51) Int. Cl. A61N 1/05; A61N 1/36

(52) U.S. Cl. 607/72; 607/116

(57) ABSTRACT

An implantable lead is provided that comprises a lead body extending along a longitudinal axis. The lead body includes a distal end and a proximal end and a lumen within the lead body. The lead also includes a header assembly provided at the distal end of the lead body. The header assembly includes a tissue engaging end. The lead also includes an electrode provided on the header assembly. The electrode is configured to deliver stimulating pulses. The lead also includes an electrode conductor provided within the lumen of the lead body and extending from the electrode to the proximal end of the lead body. An LC resonant component is provided in at least one of the lead body and the header assembly. The LC resonant component comprises a capacitor having an elongated shape that extends along the longitudinal axis of the lead body. The capacitor has a core that is located about the longitudinal axis of the lead body. The LC resonant component further comprises an inductor wire wound in multiple turns about an exterior surface of the capacitor to form an inductor.

### CROSS REFERENCE TO RELATED APPLICATIONS

This application is related to U.S. patent application Ser. No. 12/613,435, filed Nov. 5, 2009, titled “MRI-COMPATIBLE IMPLANTABLE LEAD HAVING A HEAT SPREADER AND METHOD OF USING SAME” (Attorney Docket A09P1057).

### FIELD OF THE INVENTION

The various embodiments described herein generally relate to implantable leads, and more particularly to MRI-safe implantable leads.

### BACKGROUND OF THE INVENTION

An implantable medical device is implanted in a patient to, among other things, monitor electrical activity of a heart and to deliver appropriate electrical and/or drug therapy, as required. Implantable medical devices (“IMDs”) include for example, pacemakers, cardioverters, defibrillators, implantable cardioverter defibrillators, an appetite or pain suppression device, and the like. The electrical therapy produced by an IMD may include, for example, pacing pulses, cardioverting pulses, and/or defibrillator pulses to reverse arrhythmias (e.g. tachycardias and bradycardias) or to stimulate the contraction of cardiac tissue (e.g. cardiac pacing) to return the heart to its normal sinus rhythm.

A body implantable lead forms an electrical connection between a patient's anatomy and the IMD. The lead includes a lead body comprising a tubular, flexible biocompatible, biostable insulative sheath or housing, such as formed of silicone rubber, polyurethane or other suitable polymer. One example of a lead body is a bipolar lead having a tip electrode and a ring sensing electrode. Generally bipolar leads include two coaxial conductors with insulation therebetween that are carried within the insulative housing. Another example of a lead body is a cardioverter/defibrillator lead that includes a sensing ring, a shocking right ventricle (RV) electrode, a shocking superior vena cava (SVC) electrode and a tip sensing/pacing electrode. The lead includes a multi-lumen body, each lumen of which carries a separate conductor through the lead body to each of the sensing ring, RV electrode, SVC electrode and tip electrode.

Magnetic resonance imaging (MRI) is commonly used as an efficient technique in the diagnosis of many injuries and disorders. MRI scanners provide a non-invasive method for the examination of internal structure and function. During operation, the MRI scanner creates a static magnetic field, a gradient magnetic field and a radio frequency (RF) magnetic field. The static magnetic field may have field strength of between 0.2 and 3.0 Tesla. A nominal value of 1.5 Tesla is approximately equal to 15,000 Gauss. The time varying or gradient magnetic field may have a maximum strength of approximately 40 milli-Tesla/meter. The RF magnetic field may have a frequency between 8 and 215 MHz. For example, up to 20,000 watts may be produced at 64 MHz in a static magnetic field of 1.5 Tesla.

A concern has arisen regarding the potential interaction between the MRI environment and implantable leads. In particular, implantable leads may experience RF-induced current. The RF induced current has been found to raise the temperature in the leads to undesirable levels.

Heretofore, leads have been proposed as MRI-safe. These MRI-safe leads are coupled to, or have housed therein, a discrete resonant tuning module. The resonant tuning module includes a control circuit for determining a resonance frequency of the implantable device and an adjustable impedance circuit to change the combined resonant frequency of the medical device and the lead. The resonant circuit includes an inductor (L) alone or coupled in parallel with a capacitor (C) to form a discrete LC circuit. The inductance and capacitance values of the inductor and capacitor are tuned approximately to the frequency of an expected RF magnetic field in an MRI scanner.

Using self resonant inductors alone in a distal portion of the lead has improved electrical performance. However, the resonant current induced at RF frequencies and the resistance of the conductors and the electrodes in a lead continue to cause self resonant inductors to heat, particularly in leads that utilize PEEK (i.e. Polyetheretherketones) headers.

Existing self resonant inductors use a coil structure that is sufficiently large to afford a large amount of inductance. The large amount of inductance is needed to satisfy desired impedance requirements at the RF frequencies. As the number of turns in the inductor increase, the DC resistance and RF resistance increase which then elevates component heating.

Conventional LC resonant structures couple a capacitor in parallel with the coil electrode wire that extends along the length of the lead. The coil electrode wire functions as an inductor that extends along an entire length of the lead. The amount of inductance and capacitance necessary to tune to a given resonant frequency are generally inversely related. As the inductance is increased, the capacitance can be decreased and vise versa. In the past, it has been difficult to develop an LC architecture that is able to exhibit sufficient inductance and capacitance, still fit within a lead and afford sufficient remaining room in the lead for other lead components.

Thus, it remains challenging to implement discrete LC and L circuits within leads while still meeting performance requirements. For example, circuit size is a challenge as there is a continued desire to provide circuits that are small enough to be packaged inside the distal portion of a lead, without making the LC or L circuits too small whereby they experience very localized heating.

A need remains for a self resonant inductor solution that avoids undue heating at the header assembly or along the lead. It would be further desirable to provide an improved implantable medical lead that may be operated in an MRI environment without the generation of significant heat in the lead. Furthermore, other desirable features and characteristics will become apparent from the subsequent detailed description of the invention and the appended claims, taken in conjunction with the accompanying drawings and this background of the invention.

### SUMMARY

In accordance with an embodiment, an implantable lead is provided that comprises a lead body extending along a longitudinal axis and that includes a distal end, a proximal end and a lumen within the lead body. The lead includes a header assembly provided at the distal end of the lead body. The header assembly includes a tissue engaging end. The lead also includes an electrode provided on the header assembly. The electrode is configured to deliver stimulating pulses. The lead also includes an electrode conductor provided within the lumen of the lead body and extending from the electrode to the proximal end of the lead body. An LC resonant component is provided in at least one of the lead body and the header assembly. The LC resonant component comprises a capacitor having an elongated shape that extends along the longitudinal axis of the lead body. The capacitor has a core that is located about the longitudinal axis of the lead body. The LC resonant component further comprises an inductor wire wound in multiple turns about an exterior surface of the capacitor to form an inductor.

Optionally, the LC resonant component may be located within the header assembly or at an intermediate location along a length of the lead body. The inductor wire extends concentrically about the capacitor and includes at least one insulated filar. The inductor and capacitor are connected in parallel with one another and tuned to a resonant frequency of an MR scanner.

In accordance with an embodiment, the capacitor has first and second sets of conductive plates that are arranged along the longitudinal axis of the lead body. The first and second sets of conductive plates are interleaved with one another. The conductive plates may be oriented orthogonal to the longitudinal axis of the lead body and wrapped about the longitudinal axis. Optionally, the LC resonant component may include an insulated elongated core located about the longitudinal axis of the lead body, where the conductive plates circumferentially wrap about the elongated core. The capacitor may have a tubular shape that is centered along the longitudinal axis of the lead body. The core of the capacitor may be centered along the longitudinal axis of the lead body.

Optionally, the electrode conductor may be shaped as a coiled conductor. The LC resonant component may have a lumen therethrough with the coiled conductor extending through the lumen in the LC resonant component. The parallel combination of the inductor wire and capacitor are joined in series with the coiled conductor. The inductor wire is physically separate and distinct from the electrode conductor, with the inductor wire being joined at a connecting node to the electrode conductor. The inductor wire and electrode conductor are wound in separate coil shapes having different corresponding inner diameters, turn densities along the longitudinal axis, and turn pitches oriented with respect to the longitudinal axis. At least one of the inner diameter, turn density and turn pitch of the inductor wire of the inductor differs from the inner diameter, turn density and turn pitch of the electrode conductor.

In accordance with an alternative embodiment, an implantable medical device is provided that comprises a processor, a pulse generator for generating stimulating pulses and an implantable lead. The lead comprises a lead body extending along a longitudinal axis and that includes a header assembly provided at the distal end of the lead body. The header assembly includes a tissue engaging end. The lead also includes an electrode provided on the header assembly. The electrode is configured to deliver stimulating pulses. The lead also includes an electrode conductor provided within the lumen of the lead body and extending from the electrode to the proximal end of the lead body. An LC resonant component is provided in at least one of the lead body and the header assembly. The LC resonant component comprises a capacitor having an elongated shape that extends along the longitudinal axis of the lead body. The capacitor has a core that is located about the longitudinal axis of the lead body. The LC resonant component further comprises an inductor wire wound in multiple turns about an exterior surface of the capacitor to form an inductor.

### BRIEF DESCRIPTION OF THE DRAWINGS

FIG. 1 illustrates an implanted medical system including a pacing lead formed in accordance with an exemplary embodiment.

FIG. 2 illustrates the pacing lead shown in FIG. 1 in more detail.

FIG. 3 illustrates a partial cross-sectional view of the distal end portion of the lead body and the header assembly of FIG. 2.

FIG. 4 illustrates a partial cross-section of the header assembly of FIG. 3.

FIG. 5 illustrates a partial isometric view of an LC resonant component in the header assembly of FIG. 3 in accordance with an embodiment.

FIG. 6 illustrates a side sectional view of an LC resonant component formed in accordance with an alternative embodiment.

FIG. 7 illustrates a cross-sectional graphical representation of a capacitor that is formed in accordance with an embodiment.

FIG. 8 illustrates a perspective view of a portion of the capacitor of FIG. 7.

FIG. 9 illustrates a perspective view of a portion of the inductor and a portion of the capacitor of FIG. 6.

FIG. 10 illustrates a portion of an LC resonant component with an electrode conductor extending therethrough in accordance with an embodiment.

### DETAILED DESCRIPTION

FIG. 1 illustrates an implantable medical system 10 including an implantable lead 12 formed in accordance with an exemplary embodiment. FIG. 1 depicts a chest cavity 14 in phantom, and a heart 16 within the chest cavity 14. The medical system 10 includes an implantable medical device (IMD) 18 and the lead 12, which are both implanted in the chest cavity 14. The IMD 18 includes a processor, a pulse generator for generating stimulating pulses, and electronics to detect cardiac signals. The processor determines when to deliver pacing pulses, shocking pulses, and the like.

Optionally, the medical device 18 may be implanted elsewhere, such as in the patient's abdomen, neck, pelvis regions, etc. In the illustrated embodiment, the lead 12 is a pacing and sensing lead. However, other types of leads may be used in alternative embodiments, such as neuromodulation leads, defibrillation leads, patient monitoring leads and the like. Although the following embodiments are described principally in the context of a pacemaker/defibrillator unit capable of sensing and/or pacing pulse delivery, the medical system 10 may be applied to other IMD structures. As further examples, embodiments may be implemented in leads for devices that suppress an individual's appetite, stimulate the patients nervous or muscular systems, stimulate the patient's brain functions, reduce or offset pain associated with chronic conditions and control motor skills for handicap individuals, and the like.

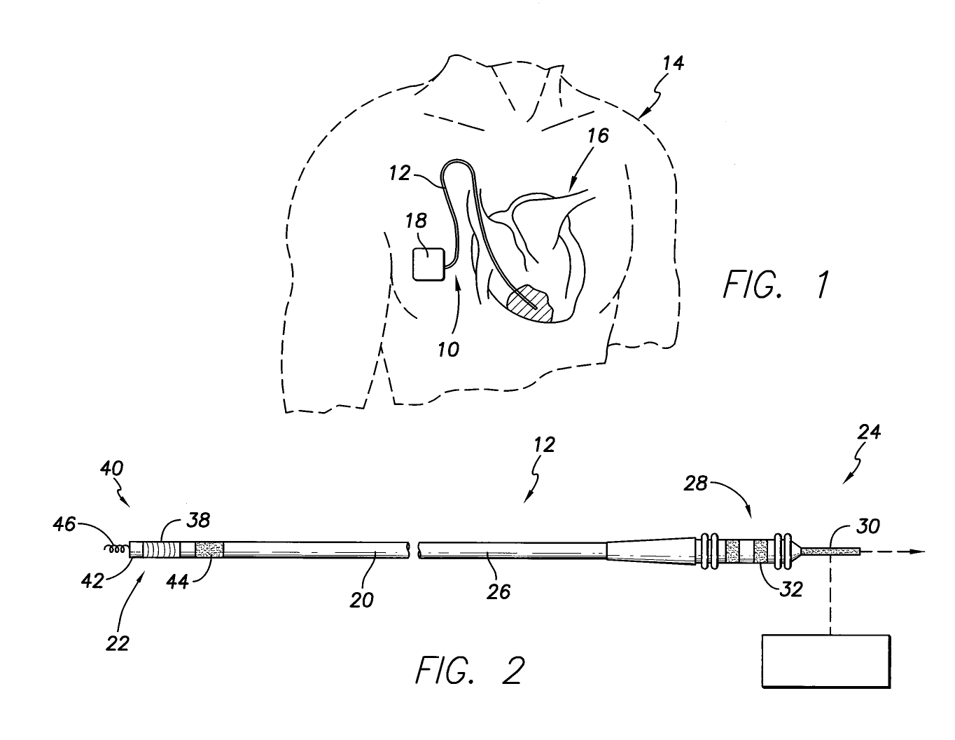

FIG. 2 illustrates the lead 12 as having an elongated lead body 20 which includes a distal end portion 22 and a proximal end portion 24. The lead body 20 has a length that extends along a longitudinal axis between the distal and proximal end portions 22 and 24. The term longitudinal axis encompasses both linear and non-linear axes. The longitudinal axis of the lead body 20 extends along a curved path that changes as the lead body 20 is flexed, bent and otherwise manipulated. The lead body 20 includes an insulating sheath 26 of a suitable insulative, biocompatible, biostable material such as, for example, PEEK (i.e. Polyetheretherketones), silicone rubber or polyurethane, extending substantially the entire length of the lead body 20.

A connector assembly 28 is provided at the proximal end portion 24 of the lead 12. The connector assembly 28 is configured to be inserted into a receiving orifice in the IMD 18. The connector assembly 28 includes first and second electrical terminals 30, 32 each being connected to respective electrical conductors, such as pacing and sensing electrical conductors, within the lead 12.

A header assembly 40 is provided at the distal end portion 22 of the lead 12. The header assembly 40 includes a tip electrode 42 at the distal end portion 22 and a ring electrode 44 proximate to the distal end portion 22. The tip electrode 42 is electrically connected to the first electrical terminal 30. The ring electrode 44 is connected to the second electrical terminal 32. In an alternative embodiment, the header assembly 40 may include only the tip electrode 42 without a corresponding ring electrode. Optionally, the header assembly 40 may include a heat spreader 38 thereabout to convey thermal energy away from the header assembly 40.

The header assembly 40 includes a fixation mechanism 46 that functions to interlock the lead 12 within the cardiac tissue at the implantation site and thereby prevent inadvertent displacement of the distal end portion 22 once the lead 12 is implanted. In the illustrated embodiment, the fixation mechanism 46 is represented by a screw-in helix that penetrates the cardiac tissue to anchor the lead 12 thereto.

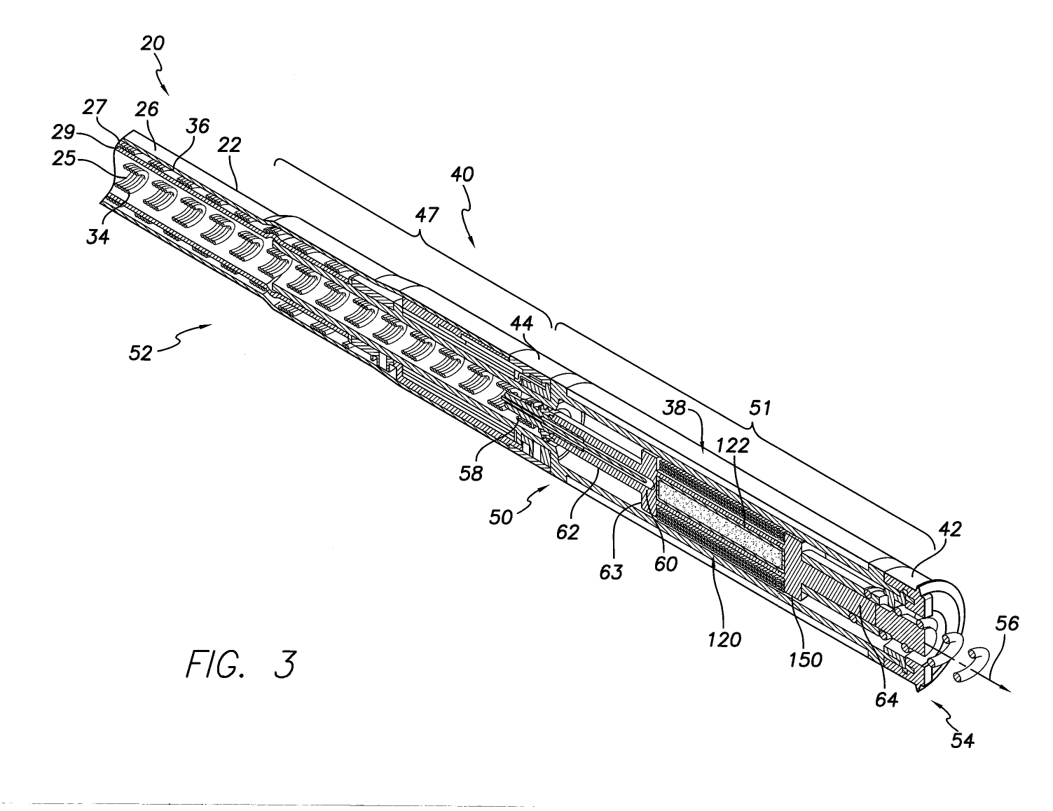

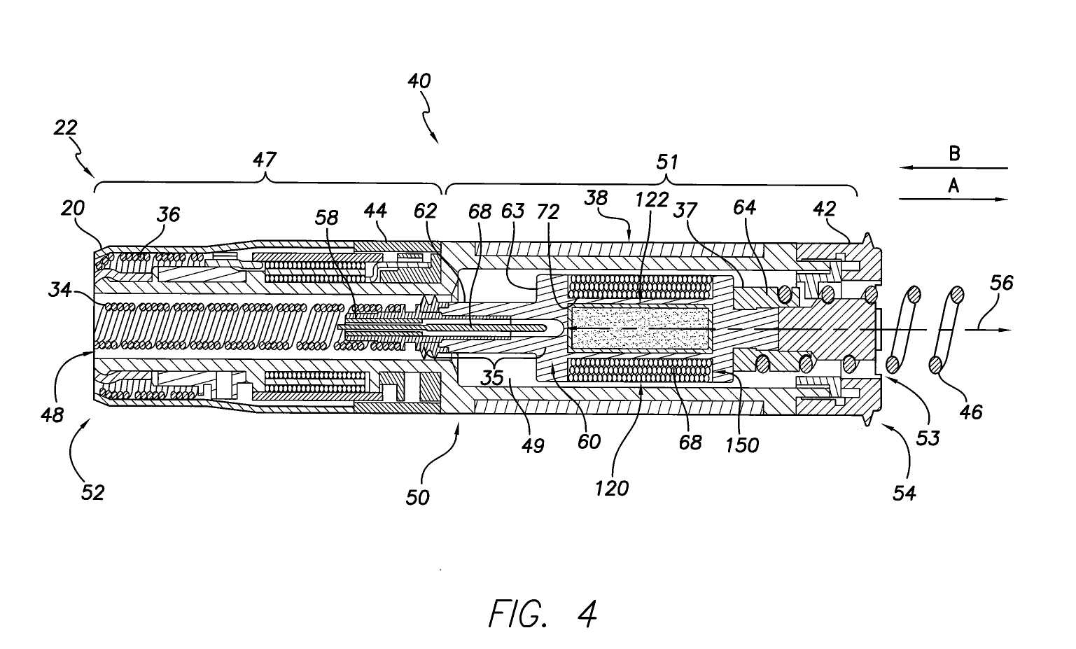

FIG. 3 illustrates a partial cross-sectional view of the distal end portion 22 of the lead body 20 and the header assembly 40 connected thereto. The lead body 20 includes an outer sheath 26 surrounding a central inner lumen 25 and an outer lumen 27. The inner and outer lumens 25 and 27 are separated by an interior wall 29. The inner and outer lumens 25 and 27, and interior wall 29 are formed concentric with one another and extend along the length of the lead body 20. The inner lumen 25 receives a coiled inner conductor 34, while the outer lumen 27 receives a coiled outer conductor 36. The inner and outer conductors 34 and 36 may each be formed of one or more filars/wires. The filars may be bare, coated with insulation or have bare segments and coated segments. For example, in one embodiment, each of the inner and outer conductors 34 and 36 may be formed from a group of 5 or 7 coated filars. The structure of the header assembly 40 is discussed below in more detail in connection with FIG. 4.

FIG. 4 illustrates a partial cross-section of the header assembly 40. The header assembly 40 includes a housing 50 that is elongated along a longitudinal axis 56. The housing 50 is a hollow, tubular element extending between a lead mating end 52 and a tissue engaging end 54. The lead mating end 52 of the housing 50 is mechanically secured to the distal end portion 22 of the lead body 20, such as by a friction fit, however, other attachment means may be used, such as adhesive, soldering, and the like. In the illustrated embodiment, the outer sheath 26 of the lead body 20 is captured between the housing 50 and a tubular insert to secure the housing 50 to the distal end portion 22 of the lead 12.

The housing 50 is formed of an insulator and is electrically inactive such that the housing 50 does not interact electrically with the cardiac tissue of the patient. Optionally, the housing 50 may be fabricated from a suitable insulative, biocompatible, biostable material. Alternatively, the housing 50 may be fabricated from a biocompatible, biostable metal or metal alloy having an insulative coating surrounding all portions of the housing 50 that may engage the cardiac tissue of the patient. Optionally, the housing 50 may include at least one fluoro-marker (not shown), or other suitable means, for identifying a position of the distal end portion 22 during and/or after implantation within the patient.

The housing 50 includes a rear section 47 and a main body 51 formed integral with one another along the axis 56. The rear section 47 includes an internal lumen 48 that is open at the lead mating end 52. The main body 51 includes a chamber 49 that is joined at one end to the internal lumen 48 and is open at the tissue engaging end 54. The tip electrode 42 is secured on the main body 51 of the housing 50 at the tissue engaging end 52. The tip electrode 42 has an opening 53 through which the fixation mechanism 46 moves. The fixation mechanism 46 of the header assembly 40 is advanced in the direction of arrow A to an extended position to penetrate, and become fixed to, the heart 16 upon implantation. The fixation mechanism 46 is retracted in the direction of arrow B until enclosed in the header assembly 40 to facilitate implantation to a desired location.

The header assembly 40 may retain various electrodes and sensors used by the implanted medical system 10 (shown in FIG. 1) for monitoring and/or pacing the heart 16 (shown in FIG. 1). For example, the header assembly 40 may include more than one ring electrode or may not include any ring electrodes. The tip electrode 42 may operate as a pacing electrode and the ring electrode 44 operates as a sensing electrode. A pacing electrode is configured to provide pacing signals to the tissue of the heart for electrically stimulating the heart tissue by delivering an electrical charge to the heart tissue. A sensing electrode is used to detect electrical activity of the heart. Optionally, the tip electrode 42 may also operate as a sensing electrode.

The rear section 47 of the housing 50 receives the inner conductor 34 within the inner lumen 48. A guide member 60 is provided within the chamber 49 of the main body 51. The guide member 60 moves in the directions of arrows A and B within the chamber 49 with the fixation mechanism 46. The guide member 60 includes a rearward extension 62, a central body 63 and a forward extension 64 arranged along the longitudinal axis 56. The central body 63 holds an LC resonant component 120. The LC resonant component 120 includes a capacitor 122 centered along the longitudinal axis 56 and an inductor wound concentrically about the capacitor 122. The rearward extension 62 holds a transition pin 58. The inner conductor 34 terminates on the transition pin 58. The transition pin 58 is connected to a segment 35 of the LC resonant component 120 that extends within the rearward extension 62. The fixation mechanism 46 is secured to and held on the forward extension 64.

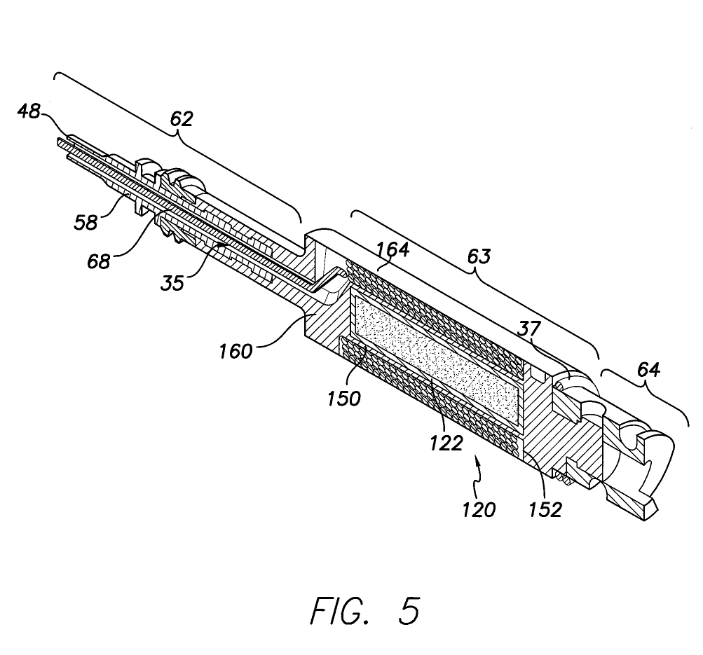

FIG. 5 illustrates a partial isometric view of the guide member 60 and LC resonant component 120. FIG. 5 better illustrates the rearward extension 62, central body 63 and forward extension 64, as well as the transition pin 58 on which the inner conductor 34 terminates. The segment 35 of a filar 68 is secured to the pin 58. The filar 68 extends through the lumen 48 (FIG. 4) in the rearward extension 62 and represents a lead end of the inductor 150 in the LC resonant component 120. For example, the filar 68 is wound concentrically about the capacitor 122 to form the inductor 150. The LC resonant component 120 includes end plates 160 and 162 that hold the capacitor 122 and the inductor 150 therebetween. An outer shell 164 extends between the end plates 160 and 162 and encloses and surrounds the inductor 150.

The capacitor 122 and the inductor 150 are electrically connected in parallel with one another to form an LC resonant circuit. The LC resonant circuit is connected in series at one end with the inner conductor 34 and at the other end with the tip electrode 42 through the segments 35 and 37, respectively. The LC resonant circuit may be tuned by setting the capacitance and inductance to desired levels. The LC resonant circuit may be tuned to a resonance frequency of 64 MHz, 128 MHz and the like, based on the MRI scanner(s) contemplated for use therewith.

Returning to FIG. 4, the housing 50 may include a heat spreader 38 that is located in a recess and wrapped about the outer wall. The heat spreader 38 is permitted to electrically float in that the heat spreader 38 is not connected to ground (ungrounded) and is not electrically connected to any of the electrodes 42 and 44, nor conductors 34 or 36. The heat spreader 38 is electrically separated from the electrodes 42 and 44, the conductors 34 and 36 and is electrically separated from the LC resonant component 120. The heat spreader 38 is located proximate to the LC resonant component 120 and is positioned at an intermediate position along the header assembly 40.

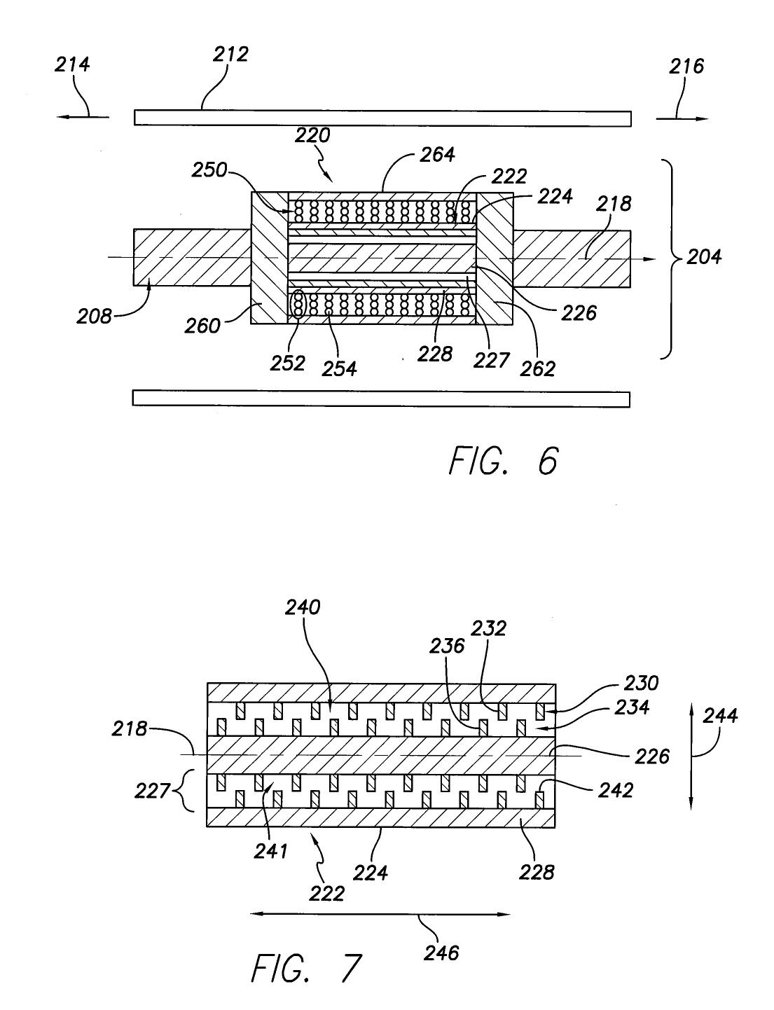

FIG. 6 illustrates a side sectional view of an LC resonant component 220 formed in accordance with an alternative embodiment. Optionally, the LC resonant component 220 may be provided within a header assembly as well. The LC resonant component 220 is provided at a discrete location along a length of a lead body 212 at an end or intermediate location within a lumen 204 of the lead body 212 such as in radial alignment with one or more electrodes. The lead body 212 extends along a longitudinal axis 218 in a direction 214 toward the distal end and in a direction 216 toward the proximal end. The lead body 212 includes a lumen 204 within the lead body 212. In the example of FIG. 6, the lead body 212 includes an electrode conductor 208 centered along the lumen 24. The electrode conductor 208 extends from an electrode to the proximal end of the lead body 212. The electrode conductor 208 may terminate at, and be electrically connected to, opposite ends of the LC resonant component 220. Optionally, the electrode conductor may be continuous and pass through a central lumen formed through the LC resonant component 220.

The LC resonant component 220 includes a cylindrical capacitor 222 having an elongated shape that extends along the longitudinal axis 218 of the lead body 212. The capacitor 222 has an inner core 226 and an outer layer 228 that are separated by a gap 227 that holds capacitor plates. An inductor 250 is arranged concentrically about outer layer 228 of the capacitor 220. The capacitor 220 and inductor 250 are held within a LC component housing that comprises end caps 260 and 262 and an outer shell 264. The end caps 260 and 262 are positioned at opposite ends of the LC resonant component 220. The outer shell 264 extends in a direction parallel to the longitudinal axis 218 between the end caps 260 and 262. The outer shell 264 is located over an outer surface of the inductor 250. The outer shell 264 circumferentially encloses and surrounds the inductor 250 and capacitor 220.

The inductor 250 is formed from an inductor wire 252 that is wound in multiple turns 256 about an exterior surface 224 of the capacitor 220. The electrode conductor 208 constitutes a coiled conductor that extends along the lead body 212. The inductor 250 and capacitor 220 are joined in parallel with one another and are joined in series with the electrode conductor 208. The inductor wire 252 is physically separate and distinct from the electrode conductor 208. The inductor wire 252 is joined at a connecting node to the electrode conductor 208.

FIG. 7 illustrates a cross-sectional graphical representation of the capacitor 222 of FIG. 6 that is formed in accordance with an embodiment. The inner core 226 is elongated and formed of an insulated material. The core 226 is concentrically located within the outer layer 228 that has the outer surface 224. The core 226 and outer layer 228 are spaced apart from one another by the gap 227. The gap 227 includes capacitor plates 232 and 236 that are separated from one another by a dielectric layer 242. The capacitor plates 232 are arranged in an outer set 230 and the capacitor plates 236 are arranged in an inner set 234. The capacitor plates 236 in the inner set 234 are distributed along a length of the capacitor 222 and are separated from one another by inter-plate spacing 241. The capacitor plates 232 in the outer set 230 are distributed along the length of the capacitor 222 and are separated from one another by inter-plate spacing 240. The inner and outer sets 234 and 230 are staggered relative to one another along the length of the capacitor 222 such that capacitor plates 232 are offset and interleaved with capacitor plates 234. Capacitor plates 232 are aligned with the inter-plate spacings 241, while capacitor plates 236 are aligned with the inter-plate spacings 240.

The capacitor 222 has an overall elongated tubular shape in the direction of arrow 246 and a circular cross-section in the direction of arrow 244. By interleaving the capacitor plates 232 and 236, and filling the gap 227 there between with dielectric material 242, the capacitor 222 is able to provide a large capacitance within a small radial form factor (in the direction of arrow 44).

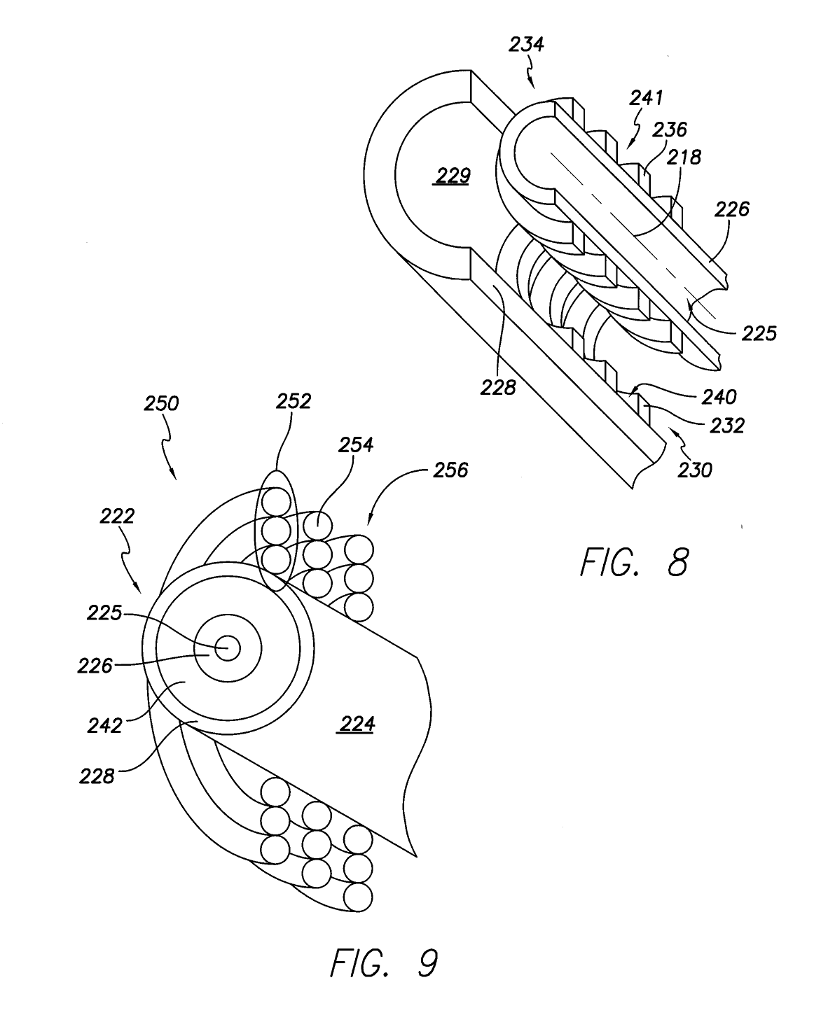

FIG. 8 illustrates a perspective view of a portion of the capacitor 222. The capacitor plates 236 of the inner set 234 wrap about an outer surface of the inner core 226. The capacitor plates 232 of the outer set 230 wrap about an inner surface 229 of the outer layer 228. Each of the capacitor plates 232 and 236 is disc-shaped and wrapped about the longitudinal axis 218. The individual capacitor plates 232 and 236 are oriented orthogonal to the longitudinal axis 218. The inner core 226 may be hollow along the longitudinal axis 218 to form a lumen 225 therein, through which one or more electrode conductors pass.

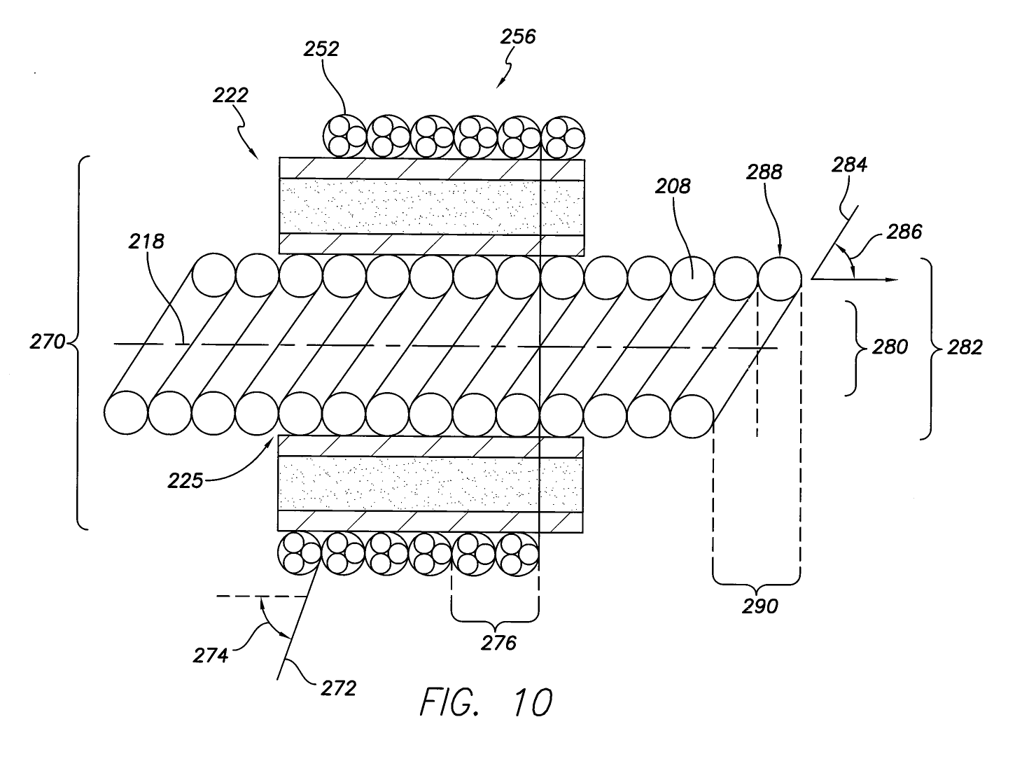

FIG. 9 illustrates a perspective view of a portion of the inductor 250 and a portion of the capacitor 222. The inductor 250 includes a multi-filar wire 252 with multiple turns 256 wrapped about the outer surface 224 of the capacitor 222. In the example of FIG. 9, the wire 252 includes three filars 254, each of which is an individually insulated conductors. Optionally, more filars or only one filar 254 may be used to form the wire 252. The number of turns 256 in the inductor 250 is varied to tune the inductance of the inductor 250 and the LC resonant component 220. The capacitance of the capacitor 220 is tuned by adjusting the number, spacing and dimensions of the capacitor plates 232 and 236. The capacitance of the capacitor 220 is also tuned by changing the material used to form the dielectric layer 242, where different materials are chosen based on the different dielectric constants.

FIG. 10 illustrates a portion of the LC resonant component 220 with the electrode conductor 208 extending therethrough. The inductor wire 252 has an inner diameter 270 that corresponds to the outer diameter of the capacitor 222. The inductor wire 252 has a turn pitch 272 which corresponds to the angular orientation 274 of the turns 256 with respect to the longitudinal axis 218. The inductor wire 252 has a turn density 276 which represents the number of turns 256 per unit of length along the lead body. In the example of FIG. 10, the turn density 276 is two turns 256 per unit of length.

The electrode conductor 208 has an inner diameter 280 and an outer diameter 282. The outer diameter 282 is smaller than the inner diameter of the lumen 225 through the LC resonant component 220. The inner and outer diameters 280 and 282 of the electrode conductor 208 are less than the inner diameter of 270 of the inductor 250. The electrode conductor 208 has a turn pitch 284 which corresponds to the angular orientation 286 of the turns 288 with respect to the longitudinal axis 218. The electrode conductor 208 has a turn density 290 which represents the number of turns 288 per unit of length along the lead body. In the example of FIG. 10, the turn density 290 is one turn 288 per unit of length. As shown in FIG. 10, the inductor wire 252 and electrode conductor 208 are wound in separate coil shapes having different corresponding inner diameters, turn densities along the longitudinal axis, and turn pitches oriented with respect to the longitudinal axis. The inductor wire 252 is distinct from the electrode conductor. At least one of the inner diameter 270, turn density 276 and turn pitch 272 of the inductor wire 252 differs from the inner diameter 280, turn density 290 and turn pitch 284 of the electrode conductor 208.

By way of example only, in one embodiment, the capacitor dimensions may be 100 mils in length and 30 mils in diameter. Optionally, the capacitor plates may not be inter-leaved with one another, such as when less capacitance is desired. Optionally, the inductor may represent a coil or spiral inductor located on a tubular shaped printed substrate. Optionally, multiple LC resonant components may be located along the length of the lead. For example, separate LC resonant components may be provided at each electrode.

By way of example, in one embodiment, the dielectric material in the capacitor may be selected to have a high dielectric constant (e.g. 20). When all or a portion of the capacitor is formed from non-bio-compatible material, a hermetic seal may be created about the capacitor, such as from a bio-compatible, non-metal moisture resistant material at the end caps and/or outer shell of the capacitor.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. An implantable lead, comprising:
a lead body extending along a longitudinal axis, the lead body including a distal end and a proximal end, and a lumen within the lead body;
a header assembly provided at the distal end of the lead body, the header assembly including a tissue engaging end;
an electrode provided on the header assembly, the electrode configured to deliver stimulating pulses;
an electrode conductor provided within the lumen of the lead body and extending from the electrode to the proximal end of the lead body; and
an LC resonant component provided in at least one of the lead body and the header assembly, the LC resonant component comprising:
a capacitor having an elongated shape that extends along the longitudinal axis of the lead body, the capacitor having a core that is located about the longitudinal axis of the lead body; and
an inductor wire wound in multiple turns about an exterior surface of the capacitor to form an inductor.

2. The implantable lead of claim 1, wherein the capacitor having first and second sets of conductive plates that are arranged along the longitudinal axis of the lead body, the first and second sets of conductive plates being interleaved with one another.

3. The implantable lead of claim 1, wherein the capacitor having conductive plates oriented orthogonal to the longitudinal axis of the lead body, the conductive plates wrapping about the longitudinal axis.

4. The implantable lead of claim 1, wherein the LC resonant component includes an insulated elongated core located about the longitudinal axis of the lead body, the capacitor including conductive plates that circumferentially wrap about the elongated core.

5. The implantable lead of claim 1, wherein the electrode conductor constitutes a coiled conductor that extends along the lead body, the LC resonant component having a lumen therethrough, the coiled conductor extending through the lumen in the LC resonant component.

6. The implantable lead of claim 1, wherein the electrode conductor constitutes a coiled conductor that extends along the lead body, the inductor wire and capacitor being joined in parallel with one another and in series with the coiled conductor.

7. The implantable lead of claim 1, wherein the capacitor has a cylindrical shape that is centered along the longitudinal axis of the lead body.

8. The implantable lead of claim 1, wherein the core of the capacitor is centered along the longitudinal axis of the lead body.

9. The implantable lead of claim 1, wherein the inductor wire is physically separate and distinct from the electrode conductor, the inductor wire being joined at a connecting node to the electrode conductor.

10. The implantable lead of claim 1, wherein the inductor wire and electrode conductor are wound in separate coil shapes having corresponding inner diameters, turn densities along the longitudinal axis, and turn pitches oriented with respect to the longitudinal axis, the inductor wire being distinct from the electrode conductor such that at least one of the inner diameter, turn density and turn pitch of the inductor wire differs from the inner diameter, turn density and turn pitch of the electrode conductor.

11. The implantable lead of claim 1, wherein the LC resonant component is located within the header assembly.

12. The implantable lead of claim 1, wherein the inductor wire extends concentrically about the capacitor.

13. The implantable lead of claim 1, wherein the inductor wire includes at least one insulated filar.

14. The implantable lead of claim 1, wherein the header assembly includes a housing with an opening at the tissue engaging end, and a fixation member provided in the chamber proximate the tissue engaging end, the resonant inductor movably located in the chamber.

15. The implantable lead of claim 1, wherein the LC resonant component is located at an intermediate position along the header assembly.

16. The implantable lead of claim 1, wherein the inductor wire and capacitor are connected in series and tuned to a resonant frequency of an MR scanner.

17. An implantable medical device, comprising:
a processor;
a pulse generator for generating stimulating pulses; and
an implantable lead, the lead comprising:
a lead body extending along a longitudinal axis, the lead body including a distal end and a proximal end and a lumen within the lead body;
a header assembly provided at the distal end of the lead body, the header assembly including a tissue engaging end;
an electrode provided on the header assembly, the electrode configured to deliver stimulating pulses;
an electrode conductor provided within the lumen of the lead body and extending from the electrode to the proximal end of the lead body; and
an LC resonant component provided in at least one of the lead body and the header assembly, the LC resonant component comprising:
a capacitor having an elongated shape that extends along the longitudinal axis of the lead body, the capacitor having a core that is located about the longitudinal axis of the lead body; and
an inductor wire wound in multiple turns about an exterior surface of the capacitor to form an inductor.

18. The implantable device of claim 17, wherein the capacitor has first and second sets of conductive plates that are arranged along the longitudinal axis of the lead body, the first and second sets of conductive plates being interleaved with one another.

19. The implantable device of claim 17, wherein the LC resonant component includes an insulated elongated core located about the longitudinal axis of the lead body, the capacitor including conductive plates that circumferentially wrap about the elongated core.

20. The implantable device of claim 17, wherein the inductor wire and electrode conductor are wound in separate coil shapes having corresponding inner diameters, turn densities along the longitudinal axis, and turn pitches oriented with respect to the longitudinal axis, the inductor wire being distinct from the electrode conductor such that at least one of the inner diameter, turn density and turn pitch of the inductor wire differs from the inner diameter, turn density and turn pitch of the electrode conductor.
